(19) United States
(12) Patent Application Publication
(10) Pub. No.: US 2021/0220384 A1
(43) Pub. Date: Jul. 22, 2021

(54) ORAL ADMINISTRABLE PHARMACEUTICAL COMPOSITION

(71) Applicant: TAIHO PHARMACEUTICAL CO., LTD., Tokyo (JP)

(72) Inventor: Yoshito OHNISHI, Tokushima-shi (JP)

(73) Assignee: TAIHO PHARMACEUTICAL CO., LTD., Tokyo (JP)

(21) Appl. No.: 17/225,232

(22) Filed: Apr. 8, 2021

Related U.S. Application Data
(63) Continuation of application No. 14/373,426, filed on Jul. 21, 2014, now abandoned.

(30) Foreign Application Priority Data
Feb. 15, 2012 (JP) 2012-031143

(51) Int. Cl. A61K 31/7072; A61K 31/513; A61K 9/16; A61K 9/20

(52) U.S. Cl. CPC A61K 31/7072; A61K 31/513; A61K 9/2054; A61K 9/2018; A61K 9/2059; A61K 9/1623

(57) ABSTRACT

The present invention provides an FTD and TPI-containing orally administrable pharmaceutical composition which can be orally administered and is stable even under high-humidity conditions. An orally administrable pharmaceutical composition which comprises α,α,α-trifluorothymidine and 5-chloro-6-(2-iminopyrrolidine-1-yl)methyl-2,4(1H,3H)-pyrimidine dione hydrochloride as active ingredients and additives having a critical relative humidity of 85% or more at 25° C. as an excipient.

### CROSS-REFERENCE TO RELATED APPLICATIONS

The present application is a continuation of and claims the benefit of priority to U.S. application Ser. No. 14/373,426, filed Jul. 21, 2014, which is the National Stage of the International Patent Application No. PCT/JP2013/053513, filed Feb. 14, 2013, which is based upon and claims the benefit of priority to Japanese Application No. 2012-031143, filed Feb. 15, 2012. The entire contents of all of the above applications are incorporated herein by reference.

### TECHNICAL FIELD

The present invention relates to an orally administrable pharmaceutical composition comprising α,α,α-trifluorothymidine (FTD) and 5-chloro-6-(2-iminopyrrolidine-1-yl)methyl-2,4(1H,3H)-pyrimidine dione hydrochloride (TPI).

### BACKGROUND ART

A combination drug comprising α,α,α-trifluorothymidine (FTD) and 5-chloro-6-(2-iminopyrrolidine-1-yl)methyl-2,4(1H,3H)-pyrimidine dione hydrochloride (TPI) is an anti-tumor agent in which FTD, which has an action for inhibiting thymidylate formation and an action for inhibiting DNA synthesis by incorporation into DNA to exert an anti-tumor effect, is combined with TPI, which has an action for inhibiting thymidine phosphorylase, to thereby suppress degradation of FTD in vivo and enhance the anti-tumor effect (Patent Literature 1).

An anti-tumor agent “TAS-102” in which FTD and TPI are combined in a molar ratio of 1:0.5 is now under development as an orally administrable formulation (Non Patent Literatures 1 and 2). As for the orally-administrable TAS-102 formulation, tablets, granules, capsules, and the like are known so far (Patent Literatures 1 and 2). However, the quality, particularly the storage stability of the formulation has not been sufficiently investigated.

In the case of formulation, in order that medicaments are orally administered with ease, excipients, binders, disintegrating agents, lubricants, taste-masking agents, and the like are usually allowed to be contained, in addition to the active ingredient. Of these, excipients are added to increase the bulk to thereby adjust the size and mass of oral medicaments to a size and mass suitable for handling and ingestion. The mass proportion of excipients often becomes large relative to the amount of medicaments. Accordingly, excipients among formulation additives have large influence on the stability of formulations, and have to be chosen with due care.

Meanwhile, in medical settings, in order to prevent accidental ingestion and to enhance medication compliance, one-dose packaging to package various medicaments into each one dosage form is promoted, and thus, stable and high-quality formulations are desired even without moisture-proof packaging. Also, if moisture-proof packaging becomes unnecessary, advantages are brought about, such as elimination of trouble of opening packages and elimination of waste packages.

### CITATION LIST

### Patent Literature

### Patent Literature 1

International Publication No. WO 96/30346

### Patent Literature 2

International Publication No. WO 2006/80327

### Non Patent Literature

### Non Patent Literature 1

International Journal of Oncology 25: 571-578, 2004

### Non Patent Literature 2

Invest New Drugs 26(5): 445-54, October 2008.

### SUMMARY OF INVENTION

### Technical Problem

The present inventor has added various formulation additives to the above FTD and TPI, and has investigated the storage stability of the resulting compositions under various conditions. Then, it has been proved that the amount of FTD and TPI related substances were increased when stored particularly under high-humidity conditions depending on types of formulation additives added.

Accordingly, an object of the present invention is to provide an FTD and TPI-containing orally administrable pharmaceutical composition which can be orally administered and whose active ingredients are stable even under high-humidity conditions.

### Solution to Problem

Thus, the present inventor has added various additives to FTD and TPI and evaluated the storage stability, and has found that a stable orally administrable pharmaceutical composition in which mass of related substances is not substantially increased even stored in the case of using a sugar having a high critical relative humidity, completing the present invention.

That is, the present invention provides an orally administrable pharmaceutical composition comprising FTD and TPI as active ingredients and a sugar having a critical relative humidity of 85% or more at 25° C. as an excipient.

Also, the present invention provides an orally administrable pharmaceutical formulation comprising the above-described orally administrable pharmaceutical composition, which is coated.

### Advantageous Effects of Invention

According to the present invention, high-quality formulations having secured formulation stability even under high-humidity conditions can be provided to patients and medical staffs.

### DESCRIPTION OF EMBODIMENTS

The active ingredients of the orally administrable pharmaceutical composition of the present invention are FTD and TPT. The molar ratio of FTD and TPT contained in the composition is preferably 1:0.5. Also, the content of FTD per dosage unit of the orally administrable pharmaceutical composition is preferably from 5 to 35 mg and more preferably from 15 to 20 mg.

Although the contents of FTD and TPI, which are the active ingredients of the oral pharmaceutical composition of the present invention, depend on formulation forms and regimens, and may be selected without particular limitation and as appropriate, the amount of each active ingredient in pharmaceutical composition is preferably from of the order of 1 to 40% by mass.

The orally administrable pharmaceutical composition of the present invention, to which sugars having a critical relative humidity of 85% or more at 25° C. as an excipient is added, suppresses increases in FTD and TPI related substances even stored under high-humidity conditions. A “critical relative humidity” herein means a well-known indicator representing the hygroscopicity, and refers to a relative humidity when a rapid increase in the amount of moisture absorbed in a sample is observed in the case where the relative humidity is increased. The critical relative humidity can be checked by measuring the change in the weight of a sample at 25° C. and a relative humidity of from 10 to 95% using, for example, a moisture sorption analyzer (DVS-1, Surface Measurement Systems Ltd.). “A critical relative humidity at 25° C. is 85% or more” means that moisture is not substantially absorbed when the relative humidity at 25° C. is less than 85%. Also, “no critical relative humidity” means that moisture is absorbed at a low humidity depending on the humidity, and a rapid increase in the amount of moisture absorbed associated with an increase in the relative humidity is not observed.

The sugar having a critical relative humidity of 85% or more at 25° C. in the oral pharmaceutical composition of the present invention is not particularly limited as long as it has critical relative humidity of 85% or more at 25° C., and examples of the sugars include monosaccharides, oligosaccharides, and sugar alcohols.

Of these sugars, from a viewpoint of the stability of the aforementioned FTD and TPI, disaccharides or sugar alcohols having a critical relative humidity of 85% or more at 25° C. are preferred, disaccharides or sugar alcohols having a critical relative humidity of 90% or more at 25° C. are more preferred, and disaccharides or sugar alcohols having a critical relative humidity of 95% or more at 25° C. are particularly preferred. Specifically, lactose (including anhydride and hydrate), sucrose, mannitol, trehalose, maltose, maltitol, or erythritol is preferred, lactose, sucrose, mannitol, trehalose, or maltose is more preferred, lactose, sucrose, or mannitol is more preferred, and lactose or mannitol is particularly preferred. It should be noted that these sugars may be used singly or in combination of two or more.

The content of the sugar having a critical relative humidity of 85% or more in the orally administrable pharmaceutical composition of the present invention is, from viewpoints of the stability of FTD and TPI and of the function as an excipient, preferably 3.6 parts by mass or more, more preferably from 3.6 to 50 parts by mass, still more preferably from 3.7 to 25 parts by mass, and particularly preferably from 3.7 to 10 parts by mass, based on 1 part by mass of FTD.

Also, disintegrating agents can be further added to the orally administrable pharmaceutical composition of the present invention in order to secure good disintegrability at oral administration. However, most disintegrating agents have no critical relative humidity, and may impair the stability of FTD and TPI depending on the types. The disintegrating agent in the orally administrable pharmaceutical composition of the present invention is, from a viewpoint of combining the stability of FTD and TPI and the disintegrability of the pharmaceutical composition, preferably low-substituted hydroxypropyl cellulose, carmellose, corn starch, partly pregelatinized starch, and crospovidone, more preferably low-substituted hydroxypropyl cellulose, carmellose, corn starch, or partly pregelatinized starch, and particularly preferably low-substituted hydroxypropyl cellulose, corn starch, or partly pregelatinized starch. These may be used singly or in combination of two or more. The content of the disintegrating agent is, from a viewpoint of combining the stability of FTD and TPI in the pharmaceutical composition of the present invention and the disintegrability of the pharmaceutical composition, preferably from 2 to 16% by mass, more preferably from 3 to 13% by mass, still more preferably from 3 to 10% by mass, and particularly preferably from 3 to 7% by mass in the total amount of the pharmaceutical composition.

Although the contents of FTD and TPI, which are the active ingredients of the orally administrable pharmaceutical composition of the present invention, depend on formulation forms and regimens, and may be selected without particular limitation and as appropriate, the amount of each active ingredient in the total amount of the pharmaceutical composition is preferably from of the order of 1 to 40% by mass. Of additives for the pharmaceutical composition, the proportion of the sugar having a critical relative humidity of 85% or more at 25° C. in the present invention is, from a viewpoint of the stability of the active ingredients, preferably from 50 to 100% by mass, more preferably a range from 70 to 100% by mass, and particularly preferably from 70 to 98% by mass, in the total amount of the additives.

Alternatively, excipients other than the sugar having a critical relative humidity of 85% or more at 25° C. may be added to the orally administrable pharmaceutical composition of the present invention. From a viewpoint of the stability of the active ingredients, the proportion of the sugar having a critical relative humidity of 85% or more at 25° C. is preferably 50% by mass or more, more preferably 70% by mass or more, more preferably 90% by mass or more, and particularly preferably 100% by mass in the total excipient.

The orally administrable pharmaceutical composition of the present invention may further contain various additives generally used, to the extent that the effects of the present invention are not prevented. Examples of the additive include, but not particularly limited to, as long as the additive is one generally used, excipients other than the aforementioned sugar having a critical relative humidity of 85% or more at 25° C., binders, lubricants, flavoring agents, colorants, and taste-masking agents.

Examples of the binder include hydroxypropyl cellulose, hypromellose, and polyvinyl alcohol. Examples of the lubricants include hydrogenated oils, sucrose fatty acid esters, and stearic acid. Examples of the colorant include food yellow No. 5, food blue No. 2, food lake, ferric oxide, yellow ferric oxide, and titanium oxide. Examples of the flavoring agent include various orange and lemon perfumes. Examples of the taste-masking agent include l-menthol, camphor, and mint. These may be used singly or in combination of two or more.

The content of the binder herein is preferably from 0.001 to 5% by mass and more preferably from 0.01 to 3% by mass in the total composition. The content of the lubricant is preferably from 0.001 to 3% by mass and more preferably from 0.01 to 2% by mass in the total composition.

Examples of the form of the orally administrable pharmaceutical composition of the present invention include granules, compression-molded products (for example, uncoated tablets), and mixtures.

Also, the orally administrable pharmaceutical composition of the present invention, from a viewpoint of securing storage stability of the active ingredients, is preferably substantially free of metal salts, such as alkali metal salts and alkaline earth metal salts. “is substantially free” herein refers to from 0 to 0.1 parts by mass, preferably from 0 to 0.05 parts by mass, more preferably from 0 to 0.01 parts by mass, and still more preferably 0 parts by mass, based on 1 part by mass of FTD.

Although the orally administrable pharmaceutical composition of the present invention may be used as it is as a pharmaceutical formulation, the formulation can be further coated on its surface to be an orally administrable pharmaceutical formulation which is stable and easily ingested. Coating herein includes film coating and sugar coating. Examples of a coating base include hypromellose, ethyl cellulose, hydroxypropyl cellulose, polyvinyl alcohol, and sucrose. It should be noted that, in the case of coating an orally administrable pharmaceutical composition comprising FTD and TPI, the coating layer may contain the aforementioned additive having a critical relative humidity less than 85% or having no critical relative humidity to the extent that the stability of FTD and TPI is not substantially influenced. Also, in the case of coating an orally administrable pharmaceutical composition containing FTD and TPI, the coating layer may contain a small amount of plasticizers, colorants, flavoring agents, taste-masking agents, and lubricants to the extent that the stability of FTD and TPI is not substantially influenced. Examples of the plasticizer include polyethylene glycol. Examples of the colorant include food tar dyes, food tar dye lakes, ferric oxide, yellow ferric oxide, and titanium oxide. Examples of the flavoring agent include various orange and lemon perfumes. Examples of the taste-masking agent include l-menthol, camphor, and mint, which may be used singly or in combination of two or more. The total amount of the coating layer herein is preferably from 1 to 5% by mass and more preferably from 2 to 4% by mass in the total formulation.

Examples of the orally administrable pharmaceutical formulation of the present invention include tablets, granules, powders, and fine granules. Examples of the tablets include chewable tablets, troches, drops, and compositions which quickly dissolve or disintegrate in the mouth cavity and can be ingested even without water, and also include effervescent tablets which are dissolved to be used at time of use. Examples of the granules, powders, and fine granules include dry syrups which are dissolved to be used at time of use, and also include powder particles which quickly dissolve in the mouth cavity and can be ingested without water.

The orally administrable pharmaceutical composition and pharmaceutical formulation of the present invention can be produced in accordance with the known method for producing orally administrable formulations. Examples of the granulation method include fluid bed granulation methods, stirring granulation methods, tumbling fluid bed granulation methods, extruding granulation methods, spray granulation methods, and crushing granulation methods, which can be used to produce granules or uncoated tablets. Also, from a viewpoint of the granulation principles, granulation methods are largely divided into the dry granulation method and the wet granulation method. From a viewpoint of the stability of FTD and TPI, the dry granulation method is preferred.

According to the present invention, adding the sugar can suppress increases in formation of related substances of FTD and TPI which are potentially formed when orally administrable pharmaceutical compositions and pharmaceutical formulations comprising FTD and TPI as active ingredients are produced. The corresponding related substances herein mean components other than FTD, TPI, and additives, and mainly refer to structurally related compounds of the corresponding two active ingredients. Specifically, the related substances are substances other than FTD, TPI, and additives which are detected when measured in accordance with Liquid Chromatography described in the Japanese Pharmacopoeia, General Tests, Physical tests, after the orally administrable pharmaceutical composition and pharmaceutical formulation of the present invention are stored under certain constant conditions.

Subsequently, aspects and preferred embodiments of the present invention are shown below.

[1] An orally administrable pharmaceutical composition comprising α,α,α-trifluorothymidine (FTD) and 5-chloro-6-(2-iminopyrrolidine-1-yl) methyl-2,4 (1H,3H)-pyrimidine dione hydrochloride (TPI) as active ingredients and a sugar having a critical relative humidity of 85% or more at 25° C. as an excipient.
[2] The orally administrable pharmaceutical composition according to [1], wherein a content of the sugar having a critical relative humidity of 85% or more at 25° C. is 3.6 parts by mass or more, preferably from 3.6 to 50 parts by mass, more preferably from 3.7 to 25 parts by mass, and still more preferably from 3.7 to 10 parts by mass, based on 1 part by mass of FTD.
[3] The orally administrable pharmaceutical composition according to [1] or [2], wherein the sugar having a critical relative humidity of 85% or more at 25° C. is disaccharide or sugar alcohol.
[4] The orally administrable pharmaceutical composition according to any of [1] to [3], wherein the sugar having a critical relative humidity of 85% or more at 25° C. is one or more selected from lactose, sucrose, mannitol, and erythritol, and preferably one or more selected from lactose, sucrose, and mannitol.
[5] The orally administrable pharmaceutical composition according to any of [1] to [4], comprising FTD and TPI at a molar ratio of 1:0.5.
[6] The orally administrable pharmaceutical composition according to any of [1] to [5], further comprising, as a disintegrating agent, one or more selected from low-substituted hydroxypropyl cellulose, carmellose, corn starch, partly pregelatinized starch, and crospovidone, preferably one or more selected from low-substituted hydroxypropyl cellulose, carmellose, corn starch, and partly pregelatinized starch, and more preferably one or more selected from low-substituted hydroxypropyl cellulose, corn starch, and partly pregelatinized starch.
[7] The oral pharmaceutical composition according to [6], wherein a content of the disintegrating agent is preferably from 2 to 16% by mass, preferably from 3 to 13% by mass, more preferably from 3 to 10% by mass, and particularly preferably from 3 to 7% by mass, in the total amount of the pharmaceutical composition.
[8] The oral pharmaceutical composition according to any of [1] to [7], wherein the pharmaceutical composition is in a formulation form of a granule, a compression-molded product, or a mixture.
[9] An orally administrable pharmaceutical formulation comprising the orally administrable composition according to any of [1] to [8], wherein the composition is coated.

### EXAMPLES

Although the present invention is described in more details hereinbelow referring to Examples, Comparative Examples, Reference Examples, and Test Examples, the present invention is not intended to be limited solely by these Examples.

### Example 1

In a mortar, 40 g of FTD and 18.84 g of TPI were mixed. In a mortar, 1.6 g of this mixture and 8 g of a lactose hydrate “Lactochem DOMO” (manufactured by DMV-Fonterra Excipients GmbH & Co) were mixed to thereby obtain a mixture (see Table 1). It should be noted that the proportion of the corresponding sugars in additives is 100% in this composition.

### Example 2

A mixture was obtained in accordance with the same method as in Example 1, except that sucrose “Granulated sugar EA” (manufactured by ENSUIKO Sugar Refining Co., Ltd.) was used instead of the lactose hydrate.

### Example 3

In a plastic bag, 105 g of FTD and 49.5 g of TPI were mixed. In a tablet crusher (manufactured by Konishi-Seisakusho Co., Ltd.), 6.0 g of this mixture and 24 g of a lactose hydrate “Lactochem DOMO” (manufactured by DMV-Fonterra Excipients GmbH & Co) were mixed. Purified water was further added to this mixture, which was granulated, and then dried in Mini Jet Oven (manufactured by TOYAMA SANGYO CO., LTD.) at 70° C. for two hours to thereby obtain granules (see Table 2). It should be noted that the proportion of the corresponding sugars in additives is 100% in this composition.

### Example 4

A granule was obtained in accordance with the same method as in Example 3, except that D-mannitol (manufactured by KYOWA HAKKO BIO CO., LTD.) was used instead of the lactose hydrate (see Table 2).

### Comparative Example 1

A mixture was obtained in accordance with the same method as in Example 1, except that crystalline cellulose “Ceolus” (manufactured by Asahi Kasei Corporation) was used instead of the lactose hydrate (see Table 1).

### Comparative Example 2

A granule was obtained in accordance with the same method as in Example 3, except that D-sorbitol (manufactured by Towa Chemical Industry Co., Ltd.) was used instead of the lactose hydrate (see Table 2).

### Comparative Example 3

A granule was obtained in accordance with the same method as in Example 3, except that xylitol (manufactured by Towa Chemical Industry Co., Ltd.) was used instead of the lactose hydrate (see Table 2).

### Reference Example 1

In a mortar, 40 g of FTD and 18.84 g of TPI were mixed to thereby obtain a mixture (see Table 1).

### Test Example 1

The critical relative humidity of additives at 25° C. shown in Tables 1 and 2 was measured using a moisture sorption analyzer (DVS-1, Surface Measurement Systems Ltd.). The results are shown in Tables 1 and 2.

### Test Example 2

The mixtures obtained in Examples 1 and 2, Comparative Example 1, and Reference Example 1 were stored at 40° C./75% R.H. for a month, and then, the mass of the related substances formed was measured in accordance with Liquid Chromatography described in the Japanese Pharmacopoeia, General Tests, Physical tests. The results are shown in Table 1. It should be noted that peaks other than those of FTD, TPI, and additives are called related substance peaks and that the total mass of the related substances refers to the sum of the mass of the related substances calculated based on the area of the active ingredients from the area of the related substance peaks.

### Test Example 3

In accordance with the method described in Test Example 2, the granules obtained in Examples 3 and 4, and Comparative Examples 2 and 3 were stored at 40° C./75% R.H. for a week, and then, the mass of the formed related substances was measured in accordance with Liquid Chromatography described in the Japanese Pharmacopoeia, General Tests, Physical tests. The results are shown in Table 2.

TABLE 1
Unit: parts by mass

| | | | Comparative | Reference |
|---|---|---|---|---|
| | Example | | Example | Example |
| | 1 | 2 | 1 | 1 |
| FTD | 10 | 10 | 10 | 10 |
| TPI | 4.71 | 4.71 | 4.71 | 4.71 |
| Lactose hydrate | 73.55 | — | — | — |
| Sucrose | — | 73.55 | — | — |
| Crystalline cellulose | — | — | 73.55 | — |
| Critical relative | 95 or more | 85 or more | Not | — |
| humidity (%, at 25° C.) | | | applicable | |
| Total mass of the | 0.19 | 0.36 | 1.64 | 0.15 |
| related substances (%) | | | | |

TABLE 2
Unit: parts by mass

| | Example | | Comparative Example | |
|---|---|---|---|---|
| | 3 | 4 | 2 | 3 |
| FTD | 10 | 10 | 10 | 10 |
| TPI | 4.71 | 4.71 | 4.71 | 4.71 |
| Lactose hydrate | 58.84 | 13 | — | — |
| D-mannitol | — | 58.84 | — | — |
| D-sorbitol | — | — | 58.84 | — |
| Xylitol | — | — | — | 58.84 |
| Critical relative | 95 or more | 95 or more | 50-60 | 75-85 |
| humidity (%, at 25° C.) | | | | |
| Total mass of the | 0.08 | 0.00 | 0.81 | 0.63 |
| related substances (%) | | | | |

As clearly seen from Table 1, the total mass of the related substances of Examples 1 and 2 in which a sugar having a critical relative humidity of 85% or more at 25° C. was used as the excipient showed virtually no difference compared to Reference Example 1, and was very stable compared to Comparative Example 1. Also, from Table 2, the total mass of the related substances of Examples 3 and 4 in which a sugar having a critical relative humidity of 85% or more at 25° C. was used as the excipient was clearly less than that of Comparative Examples 2 and 3 in which a sugar having a critical relative humidity of less than 85% at 25° C. was used as the excipient, and was very stable.

From the above-described result, it was found that FTD and TPI-containing formulations having high stability even under severe conditions such as 40° C./75% R.H. can be obtained by using a sugar having a critical relative humidity of 85% or more at 25° C. as the excipient. Since formation of related substances is suppressed, it is possible to provide patients and medical staffs with formulations of higher quality.

### Example 5

In a plastic bag, 400 g of FTD, 188.4 g of TPI, 1511.6 g of a lactose hydrate, 300 g of carmellose “NS-300” (manufactured by GOTOKU CHEMICAL COMPANY LTD), and 40 g of stearic acid were mixed. This mixture was tableted with a rotary tableting machine into tablets having a diameter of 15 mm and a mass of 800 mg. Then, the tablets were crushed with a crusher to thereby obtain a granule. To 122 parts of this granule, 1 part of stearic acid was further added and mixed in a plastic bag. Uncoated tablets having a diameter of 7 mm and a mass of 123 mg were obtained by use of a rotary tableting machine (see Table 3).

### Example 6

In a mortar, 1 g of a mixture of 1 part of FTD and 0.471 parts of TPI, 6 g of a lactose hydrate, and 1 g of carmellose were mixed. From this mixture, uncoated tablets having a mass of 235.36 mg were obtained by use of a hydraulic press (see Table 3).

### Example 7

In a plastic bag, 1200 g of FTD, 565.2 g of TPI, 7258.8 g of a lactose hydrate, 480 g of partly pregelatinized starch “PCS(PC-10)” (manufactured by Asahi Kasei Chemicals Corporation), and 96 g of stearic acid were mixed. From this mixture, uncoated tables having a diameter of 7 mm and a mass of 120 mg were obtained by use of a rotary tableting machine (see Table 3).

### Example 8

In accordance with the method described in Example 7, 100 g of FTD, 47.1 g of TPI, 371.9 g of a lactose hydrate, 100 g of partly pregelatinized starch, and 6 g of stearic acid were mixed in a plastic bag. From this mixture, uncoated tablets having a diameter of 7 mm and a mass of 125 mg were obtained by use of a rotary tableting machine (see Table 4).

### Example 9

In accordance with the method described in Example 7, 100 g of FTD, 47.1 g of TPI, 371.9 g of a lactose hydrate, 25 g of partly pregelatinized starch, and 6 g of stearic acid were mixed in a plastic bag. From this mixture, uncoated tablets having a diameter of 7 mm and a mass of 110 mg were obtained by use of a rotary tableting machine (see Table 4).

### Example 10

In according with the method described in Example 7, 100 g of FTD, 47.1 g of TPI, 371.9 g of a lactose hydrate, 50 g of partly pregelatinized starch, and 6 g of stearic acid were mixed in a plastic bag. From this mixture, uncoated tablets having a diameter of 7 mm and a mass of 115 mg were obtained by use of a rotary tableting machine (see Table 4).

### Example 11

In accordance with the method described in Example 7, 100 g of FTD, 47.1 g of TPI, 521.9 g of a lactose hydrate, 75 g of partly pregelatinized starch, and 6 g of stearic acid were mixed in a plastic bag. From this mixture, uncoated tablets having a diameter of 7 mm and a mass of 150 mg were obtained by use of a rotary tableting machine (see Table 4).

### Example 12

In accordance with the method described in Example 7, 100 g of FTD, 47.1 g of TPI, 671.9 g of a lactose hydrate, 75 g of partly pregelatinized starch, and 6 g of stearic acid were mixed in a plastic bag. From this mixture, uncoated tablets having a diameter of 7 mm and a mass of 150 mg were obtained by use of a rotary tableting machine (see Table 4).

TABLE 3
Unit: parts by mass

| | Example | | |
|---|---|---|---|
| | 5 | 6 | 7 |
| FTD | 1 | 1 | 1 |
| TPI | 0.47 | 0.47 | 0.47 |
| Lactose hydrate | 3.78 | 8.83 | 6.05 |
| Carmellose | 0.75 | 1.47 | — |
| Partly pregelatinized | — | | 0.4 |
| starch | | | |
| Stearic acid | 0.15 | — | 0.08 |
| Total | 6.15 | 11.77 | 8 |

TABLE 4
Unit: parts by mass

| | Example | | | | |
|---|---|---|---|---|---|
| | 8 | 9 | 10 | 11 | 12 |
| FTD | 1 | 1 | 1 | 1 | 1 |
| TPI | 0.471 | 0.471 | 0.471 | 0.471 | 0.471 |
| Lactose hydrate | 3.719 | 3.719 | 3.719 | 5.219 | 6.719 |
| Partly pregelatinized | 1 | 0.25 | 0.5 | 0.75 | 0.75 |
| starch | | | | | |
| Stearic acid | 0.06 | 0.06 | 0.06 | 0.06 | 0.06 |
| Total | 6.25 | 5.5 | 5.75 | 7.5 | 9 |

### Example 13

In a mortar, 1 g of FTD, 0.471 g of TPI, 3.779 g of a lactose hydrate, and 0.15 g of stearic acid were mixed. From this mixture, uncoated tablets having a mass of 108 mg were obtained by use of a hydraulic press (see Table 5).

### Example 14

In a plastic bag, 1 g of FTD, 0.471 g of TPI, 3.779 g of a lactose hydrate, 0.75 g of carmellose as a disintegrating agent, and 0.15 g of stearic acid were mixed. From this mixture, uncoated tablets having a mass of 123 mg were obtained by use of a hydraulic press (see Table 5).

### Comparative Example 4

In accordance with the method described in Example 14, 0.75 g of carmellose calcium “E.C.G-505” (manufactured by GOTOKU CHEMICAL COMPANY LTD.) was used as a disintegrating agent instead of carmellose to thereby obtain uncoated tablets having a mass of 123 mg (see Table 5).

### Comparative Example 5

In accordance with the method described in Example 14, 0.75 g of croscarmellose sodium “Ac-Di-Sol” (manufactured by Asahi Kasei Corporation) was used as a disintegrating agent instead of carmellose to thereby obtain uncoated tablets having a mass of 123 mg (see Table 5).

### Test Example 4

In accordance with the method described in Test Example 2, tablets obtained in Examples 13 and 14 and Comparative Examples 4 and 5 were stored at 40° C./75% R.H. in open conditions for one month, and then, the total mass of the related substances was measured (see Table 5). As the result, even if carmellose, which is a disintegrating agent having no critical relative humidity, was contained, it was found that the disintegrability as orally administrable tablets was sufficiently secured, noticeable increases in related substances were not observed, and the storage stability was secured. In contrast, if carmellose calcium or croscarmellose sodium was contained as a disintegrating agent, the mass of related substances was noticeably increased, and the storage stability was not secured.

TABLE 5
Unit: parts by mass

| | Example | | Comparative Example | |
|---|---|---|---|---|
| | 13 | 14 | 4 | 5 |
| FTD | 1 | 1 | 1 | 1 |
| TPI | 0.471 | 0.471 | 0.471 | 0.471 |
| Lactose hydrate | 3.779 | 3.779 | 3.779 | 3.779 |
| Carmellose | — | 0.75 | — | — |
| Carmellose calcium | — | — | 0.75 | — |
| Croscarmellose | — | — | — | 0.75 |
| sodium | | | | |
| Stearic acid | 0.15 | 0.15 | 0.15 | 0.15 |
| Total | 5.4 | 6.15 | 6.15 | 6.15 |
| Total mass of the | 0.286 | 0.404 | 1.194 | 2.529 |
| related substances | | | | |
| (%) | | | | |

### Example 15

In accordance with the method described in Example 7, 50 g of FTD, 23.55 g of TPI, 226.45 g of a lactose hydrate, and 3 g of stearic acid were mixed in a plastic bag. From this mixture, uncoated tablets having a mass of 121.2 mg were obtained by use of a rotary tableting machine (see Table 6).

### Example 16

In accordance with the method described in Example 7, 50 g of FTD, 23.55 g of TPI, 211.45 g of a lactose hydrate, 15 g of a disintegrating agent (any of corn starch “corn starch W” (manufactured by NIHON SHOKUHIN KAKO CO., LTD.), partly pregelatinized starch, or low-substituted hydroxypropyl cellulose), and 3 g of stearic acid were mixed in a plastic bag. From this mixture, uncoated tablets having a mass of 121.2 mg were obtained by use of a rotary tableting machine (see Table 6).

### Example 17

In accordance with the method described in Example 16, 50 g of FTD, 23.55 g of TPI, 196.45 g of a lactose hydrate, 30 g of a disintegrating agent (any of corn starch, partly pregelatinized starch or low-substituted hydroxypropyl cellulose), and 3 g of stearic acid were mixed in a plastic bag. From this mixture, uncoated tablets having a mass of 121.2 mg were obtained by use of a rotary tableting machine (see Table 6).

### Test Example 5

In accordance with the method described in Test Example 2, tablets obtained in Examples 15, 16, and 17 were stored at 40° C./75% R.H. in open conditions for two weeks, and then, the total mass of the related substances was measured (see Table 6).

As the result, noticeable increases in related substances were not observed in any of the disintegrating agents and amounts thereof.

TABLE 6
Unit: parts by mass

| | Example | | | | | | |
|---|---|---|---|---|---|---|---|
| | 15 | 16 | | | 17 | | |
| FTD | 1 | 1 | 1 | 1 | 1 | 1 | 1 |
| TPI | 0.417 | 0.471 | 0.471 | 0.471 | 0.471 | 0.471 | 0.471 |
| Lactose hydrate | 4.529 | 4.229 | 4.229 | 4.229 | 3.929 | 3.929 | 3.929 |
| Corn starch | — | 0.3 | — | — | 0.6 | — | — |
| Partly | — | — | 0.3 | — | — | 0.6 | — |
| pregelatinized | | | | | | | |
| starch | | | | | | | |
| Low-substituted | — | — | — | 0.3 | — | — | 0.6 |
| hydroxypropyl | | | | | | | |
| cellulose | | | | | | | |
| Stearic acid | 0.06 | 0.06 | 0.06 | 0.06 | 0.06 | 0.06 | 0.06 |
| Total | 6.06 | 6.06 | 6.06 | 6.06 | 6.06 | 6.06 | 6.06 |
| Total mass of | 0.188 | 0.2 | 0.266 | 0.332 | 0.282 | 0.334 | 0.391 |
| the related | | | | | | | |
| substances (%) | | | | | | | |

1. An oral pharmaceutical composition, comprising:
α,α,α-trifluorothymidine;
5-chloro-6-(2-iminopyrrolidine-1-yl) methyl-2,4 (1H,3H)-pyrimidine dione hydrochloride;
at least one substance selected from the group consisting of lactose, sucrose, and mannitol; and
a partly pregelatinized starch included in an amount of from 3 to 10% by mass with respect to a total amount of the oral pharmaceutical composition,
wherein the oral pharmaceutical composition does not include corn starch.

2. The oral pharmaceutical composition according to claim 1, wherein the at least one substance is included in an amount of 3.6 parts by mass or more based on 1 part by mass of α,α,α-trifluorothymidine.

3. The oral pharmaceutical composition according to claim 1, wherein the at least one substance is lactose.

4. The oral pharmaceutical composition according to claim 1, wherein the at least one substance is mannitol.

5. The oral pharmaceutical composition according to claim 1, wherein α,α,α-trifluorothymidine and 5-chloro-6-(2-iminopyrrolidine-1-yl)methyl-2,4(1H,3H)-pyrimidine dione hydrochloride are included at a molar ratio of 1:0.5.

6. The oral pharmaceutical composition according to claim 1, wherein the partly pregelatinized starch is included in an amount of from 3 to 7% by mass with respect to the total amount of the oral pharmaceutical composition.

7. The oral pharmaceutical composition according to claim 1, which is in a form of a granule, a compression-molded product, or a mixture.

8. An oral pharmaceutical formulation, comprising:
the oral pharmaceutical composition according to claim 1; and
a coating formed on the oral pharmaceutical composition.

9. An oral pharmaceutical formulation, comprising:
the oral pharmaceutical composition according to claim 1; and
a coating formed on the oral pharmaceutical composition and comprising at least one of a plasticizer, colorant, flavoring agent, taste-masking agent, and lubricant.

10. The oral pharmaceutical composition according to claim 2, wherein the partly pregelatinized starch is included in an amount of from 3 to 7% by mass with respect to the total amount of the oral pharmaceutical composition.

11. The oral pharmaceutical composition according to claim 1, wherein the at least one substance is included in an amount of from 3.7 to 10 parts by mass or more based on 1 part by mass of α,α,α-trifluorothymidine.

12. The oral pharmaceutical composition according to claim 1, wherein the oral pharmaceutical composition does not include an excipient other than the at least one substance.

13. The oral pharmaceutical composition according to claim 1, wherein the at least one substance is lactose hydrate.

14. The oral pharmaceutical composition according to claim 13, wherein α,α,α-trifluorothymidine and 5-chloro-6-(2-iminopyrrolidine-1-yl)methyl-2,4(1H,3H)-pyrimidine dione hydrochloride are included at a molar ratio of 1:0.5.

15. The oral pharmaceutical composition according to claim 14, which is an uncoated tablet.

16. The oral pharmaceutical composition according to claim 14, wherein lactose hydrate is included in an amount of from 3.7 to 10 parts by mass or more based on 1 part by mass of α,α,α-trifluorothymidine.

17. The oral pharmaceutical composition according to claim 16, wherein the partly pregelatinized starch is included in an amount of from 3 to 7% by mass with respect to the total amount of the oral pharmaceutical composition.

18. The oral pharmaceutical composition according to claim 4, wherein α,α,α-trifluorothymidine and 5-chloro-6-(2-iminopyrrolidine-1-yl)methyl-2,4(1H,3H)-pyrimidine dione hydrochloride are included at a molar ratio of 1:0.5.

19. The oral pharmaceutical composition according to claim 12, wherein α,α,α-trifluorothymidine and 5-chloro-6-(2-iminopyrrolidine-1-yl)methyl-2,4(1H,3H)-pyrimidine dione hydrochloride are included at a molar ratio of 1:0.5.

20. The oral pharmaceutical formulation according to claim 8, wherein the oral pharmaceutical composition comprises α,α,α-trifluorothymidine and 5-chloro-6-(2-iminopyrrolidine-1-yl)methyl-2,4(1H,3H)-pyrimidine dione hydrochloride at a molar ratio of 1:0.5.

21. The oral pharmaceutical formulation according to claim 9, wherein the oral pharmaceutical composition comprises α,α,α-trifluorothymidine and 5-chloro-6-(2-iminopyrrolidine-1-yl)methyl-2,4(1H,3H)-pyrimidine dione hydrochloride at a molar ratio of 1:0.5.

22. The oral pharmaceutical composition according to claim 1, wherein the at least one substance is included in an amount of 70 to 98% by mass with respect to a total amount of an additive.

23. The oral pharmaceutical composition according to claim 14, wherein the at least one substance is included in an amount of 70 to 98% by mass with respect to a total amount of an additive.

24. The oral pharmaceutical composition according to claim 17, further comprising:
stearic acid in an amount of from 0.01 to 2% by mass with respect to the total amount of the oral pharmaceutical composition.

25. The oral pharmaceutical composition according to claim 24, wherein the amount of lactose hydrate is 70 to 98% by mass with respect to a total amount of an additive in the composition, and the amount of lactose hydrate, the amount of stearic acid, and the amount of pregelatinized starch count towards the total amount of the additive.

26. The oral pharmaceutical composition according to claim 25, wherein the oral pharmaceutical composition does not include an additive other than lactose hydrate, stearic acid and pregelatinized starch.
